(19) United States
(12) Patent Application Publication
(10) Pub. No.: US 2010/0330604 A1
(43) Pub. Date: Dec. 30, 2010

(54) QUANTIFICATION OF CHANGES IN THE DEGREES OF ORDER OF CELLULAR AND VIRAL MEMBRANES AND APPLICATIONS TO DIAGNOSIS, TREATMENT AND DRUG SCREENING

(76) Inventors: Benoit Labarthe, Montreal (CA); Ange Maguy, Outremont (CA); Arnaud Bonnefoy, Montreal (CA); Pierre Theroux, Montreal (CA)

Correspondence Address: Tessier & Associes, C P 54029, Mont-Royal, QC, H3P 3H4, CA

(21) Appl. No.: 12/733,474

(22) PCT Filed: Sep. 5, 2008
(86) PCT No.: PCT/CA2008/001625

§ 371 Date: Sep. 7, 2010

Related U.S. Application Data
(60) Provisional application No. 60/935,871, filed on Sep. 5, 2007.
(60) Provisional application No. 61/129,036, filed on Jun. 2, 2008.

(51) Int. Cl. C12Q 1/02

(52) U.S. Cl. 435/29

(57) ABSTRACT

A method for characterizing cell membrane order in a cell. The method includes: staining the cell with di-4-ANEPPDHQ to produce a stained cell; irradiating the stained cell with an excitation light, the excitation light being capable of inducing fluorescence in the di-4-ANEPPDHQ; measuring a fluorescence spectrum of the stained cell; and characterizing the cell membrane order by computing a spectral signature of the stained cell from the fluorescence spectrum, the spectral signature providing a character of the cell membrane order.

### FIELD OF THE INVENTION

The present invention relates to the quantification of changes in the degree of order of cellular and viral membranes and to applications to diagnostic and drug screening. More specifically, the present invention is concerned with a method of diagnosis, follow-up of treatment, and of screening of drugs by detection and quantification of changes in the degree of order of the cellular and viral membranes by means of a fluorescent sensor and of an optical detection.

### BACKGROUND OF THE INVENTION

Singer and Nicolson described for the first time the plasmic membrane as a fluid mosaic in 1972[4]. Later studies introduced the concept of lipid organization and order of the membrane defining specific zones in the plasmic membrane [3].

The fluidity and the state of order of the membrane depend on the nature and relative proportions of the constituents of the membrane (lipids, proteins, sugars), of its conformation (thickness of the lipid bilayer, incurvation) [5], of its relation with the cytoskeleton [6], of the extracellular environment (extracellular matrix [7], hemodynamic conditions) and consequently of cellular activity [8]. These parameters are also modifiable by variations in temperature and pressure [9, 10]. Numerous biological or pathological phenomena and effects of treatments have been shown to be related to modifications in the degree of membrane order [11, 12].

Lipidic ordered domains are also called membrane rafts or lipid microdomains. There are several categories of rafts [8, 13]. The study of rafts is rapidly growing in many fields of human pathology, for example in the following fields.

In cardiology, in cardiomyocytes, lipid rafts are involved in the function of ionic channels [15] and in the signalling of G proteins [16]. Lipid rafts play an important role during platelet activation [12] and the coagulation involved in thrombus formation [17, 18] in in vitro or ex vivo studies. Inhibitors of the HMG-CoA reductase, also named statins, are hypothesized to modify the regulation of the rafts functions [19] by destabilizing the membrane of cells. [20, 21] These studies are still marginal and none analysed the membrane structure of cell samples from patients treated by a statin, certainly because of the complexity of and the time required to perform current techniques directed to study of membrane structure.

In infectiology, lipid rafts were studied in the context of infection by the human immunodeficiency virus (HIV). Indeed the virus entry into CD4 lymphocytes during the cell infection is dependent on rafts, as well as the formation of the viral particles from the membrane of the cell host. [22] Lipid rafts are also involved for example in infections by Pseudomonas aeruginosa, which is the causal agent of a large portion of the opportunist and hospital-borne infections in immunodepressed patients. Indeed, treating infected patients with a drug destroying lipid rafts decreases the attachment of the bacterium in vitro, and also decreased the in vivo infections of mice by Pseudomonas aeruginosa. [22] Infection by prion proteins also involves these orderly structures. The bundling of PrPc proteins initiating intracellular signalling is indeed dependent on their localization in lipid rafts. [25, 26]

In immunology, lipid rafts have been shown to be involved in the modulation of the activity of T lymphocytes and the formation of immunological synapses. Statins have been shown to modulate the activity of T lymphocytes by a lipid rafts dependant mechanism in patients with systemic lupus erythematosus [30].

In cancerology, it was shown that the inhibition of squalene synthase, which is implied in the biosynthesis of the cholesterol located in lipid rafts, modulates the proliferation of cancer cells. [31]

In reproduction biology, several studies are suggesting that the composition of lipid rafts in the membrane of spermatozoids could modulate spermatozoid motility, spermatozoid capacity to penetrate into the pellucid zone and to interact with the ovule and, in a general way, the capacitation of the spermatozoid. [25, 32]

Current techniques used for the study of the ordered membrane structures are of two types: (1) isolation of fractions of membranes resistant to chemical detergent (Detergent Resistant Membrane or DRM), followed by a separation on a density gradient and biochemical analysis (example: electrophoresis and Western Blot), these DRMs being commonly considered to be lipid rafts; and (2) staining and visualization by optical or electronic microscopy of defined constituents (lipids, proteins, sugars) of rafts or non-raft fractions.

The first method is long and complex. Furthermore it was shown that detergents (particularly the Triton X-100 commonly used in many protocols) could induce the fusion of various types of membrane ordered structures [3, 8]. The results are very dependent on experimental conditions of extraction, such as, for example temperature and the nature and concentration of detergent [8, 39].

The second method allows a visualization of the lipid rafts on cells fixed by an aldehyde or an alcohol, but does not allow quantification [40]. This techniques requires a chemical fixation of the membrane, which is known to alter its structure, and is associated with a specific detection of a lipid (for example the GM1 ganglioside) or of a protein (for example the caveolin or the CD36 in platelets).

In conclusion, quantification of the degree of order of cellular and viral membranes has many applications to diagnosis, treatment and drug screening. However, current techniques used to study the degree of order in lipidic mono- and bi-layers are deficient.

Against this background, there exists a need in the industry to provide novel method for characterizing the degree of order in cellular and viral membranes.

The present description refers to a number of documents, the content of which is herein incorporated by reference in their entirety.

### OBJECTS OF THE INVENTION

An object of the present invention is therefore to provide novel method for characterizing the degree of order in cellular and viral membranes.

### SUMMARY OF THE INVENTION

In a first broad aspect, the invention provides a method for characterizing cell membrane order in a cell. The method includes: staining the cell with di-4-ANEPPDHQ to produce a stained cell; irradiating the stained cell with an excitation light, the excitation light being capable of inducing fluorescence in the di-4-ANEPPDHQ; measuring a fluorescence spectrum of the stained cell; and characterizing the cell membrane order by computing a spectral signature of the stained cell from the fluorescence spectrum, the spectral signature providing a character of the cell membrane order.

The character of the cell membrane order is either a quantitative character or a qualitative character. Examples of such characters are provided hereinbelow.

Advantageously, the invention suggests a novel approach to determine and quantify any alteration of the membrane order of cellular, viral and lipid membranes leading to extensive possibilities regarding drug screening patient monitoring and diagnosis.

In some embodiments of the invention, the fluorescence spectrum includes a first spectral band and a second spectral band and computing the spectral signature including computing a first intensity of the fluorescence spectrum in the first spectral band and a second intensity of the fluorescence spectrum in the second spectral band. In these embodiments, changes in the first and second intensities provides a quantitative characterization of cell membrane order. For example, this is done when computing the spectral signature includes computing a ratio between the first intensity and the second intensity.

The first spectral band is centered on a first central wavelength and the second spectral band is centered on a second central wavelength. In a specific embodiment of the invention, the first central wavelength is comprised in an interval of from about 500 nm to about 600 nm and the second central wavelength is comprised in an interval of from about 650 nm to about 750 nm. In a very specific embodiment of the invention, it was found that having the first central wavelength of about 575 nm and the second central wavelength of about 675 nm produced useful results.

Typically, the first spectral band and the second spectral band have a first bandwidth and a second bandwidth, respectively, the first bandwidth and the second bandwidth being of about 25 nm to about 100 nm. In a very specific embodiment of the invention, it was found that having the first bandwidth and the second bandwidth each of about 50 nm produced useful results.

It was found that unexpectedly, the fluorescence spectrum of di-4-ANEPPDHQ is characterizable as containing a number of spectral peaks, and not only arbitrary bands of fluorescence, which provides additional information useful for characterizing the cell membrane order. In a specific example of the invention, these peaks are used to divide the fluorescence spectrum in five spectral bands centered respectively on a respective central wavelength of about 530 nm, about 560 nm, about 585 nm, about 615, nm and about 645 nm and having respective bandwidths of about 30 nm, about 30 nm, about 20 nm, about 30 nm and about 30 nm, the fluorescence spectrum defining also a sixth spectral band including wavelengths longer or equal than about 660 nm, the five spectral bands and the sixth spectral band defining together six spectral bands. In these embodiments, computing the spectral signature includes computing a respective intensity of the fluorescence spectrum in at least three of the six spectral bands.

Computing the intensity in more than 2 spectral bands is not only indicative of a quantitative character of cell membrane order, but also indicative of a qualitative character of cell membrane order. Indeed, it was observed that the various spectral bands described hereinabove can vary relatively to each other in many different manners. Some modifications to the cell membrane order affect some of the spectral bands and other modifications to the cell membrane order affect other spectral bands.

In a variant, computing the spectral signature includes computing a ratio between two of the respective intensities of the fluorescence spectrum in the at least three of the six spectral bands.

For example, computing the spectral signature includes computing a respective intensity of the fluorescence spectrum in all of the six spectral bands and computing the spectral signature includes computing pairwise ratios between respective intensities of the fluorescence spectrum in the six spectral bands, for instance by computing all pairwise ratios between respective intensities of the fluorescence spectrum in the six spectral bands.

The terminology intensity relates to any measure of fluorescence energy. Examples of such measures include, non-limitatively, a mean fluorescence intensity in a spectral band, a total fluorescence energy in a spectral band, and a median fluorescence intensity in a spectral band.

In another embodiment of the invention, computing the spectral signature including deconvoluting the fluorescence spectrum to obtain a set of spectral peaks and parameterizing the set of spectral peaks.

In some embodiments of the invention, staining the cell with di-4-ANEPPDHQ to produce the stained cell includes suspending the cell in a suspension solution; and mixing the suspension solution with the cell contained therein with the di-4-ANEPPDHQ to obtain a staining solution.

For example, the di-4-ANEPPDHQ has a concentration of about 0.025 μM to about 100 μM in the staining solution, and in some advantageous specific examples, the di-4-ANEPPDHQ has a concentration of about 1 μM to about 50 μM in the staining solution.

In some embodiments of the invention, staining the cell with di-4-ANEPPDHQ to produce the stained cell further includes incubating the staining solution.

For example, incubating the staining solution includes incubating the staining solution at a temperature of about 4 C to about 60 C for a duration of about 1 minute to about 600 minutes, and in an advantageous specific example, incubating the staining solution includes incubating the staining solution at a temperature of about 4 C to about 40 C for about 5 minutes to about 60 minutes.

The above-mentioned incubation parameters and di-4-ANEPPDHQ concentrations have been found advantageous in cases in which a flow cytometer is used to measure the fluorescence spectrum. Use of a flow cytometer allows for achieving relatively large throughput in cell characterization.

In some embodiments of the invention, the cell is selected from the group consisting of blood platelets, red blood cells, neutrophils, endothelial cells, cardiomyocytes, HL1 cells, HEK 293 cells, monocytes and lymphocytes, but other cells are usable in alternative embodiments of the invention.

In some embodiments of the invention, irradiating the stained cell with the excitation light includes irradiating the stained cell with laser light having a wavelength between about 400 nm and about 500 nm. In a specific example, irradiating the stained cell with the excitation light includes irradiating the stained cell with laser light having a wavelength of about 488 nm.

In some embodiments of the invention, the spectral signature is indicative a cholesterol content of a membrane of the cell. In other embodiments, the spectral signature is indicative of a lipid and protein content of a membrane of the cell.

In some embodiments of the invention, the cell is classifiable as belonging to a specific cell category selected from a set of predetermined cell categories, the method further comprising classifying the cell as belonging to the specific cell category on a basis of the spectral signature. For example, the set of predetermined cell categories includes cell categories indicative of a cholesterol content in the cell. In another example, the cell is a blood platelet, the set of predetermined cell categories includes cell categories indicative of a coagulation activity of the platelets. In yet another example, the set of predetermined cell categories includes cell categories indicative of an apoptosis status of the cell. In yet another example, the set of predetermined cell categories includes sub-populations of cells of a predetermined type.

In another broad aspect, the invention provides a method for assessing an effect of a treatment in a subject, the treatment influencing target cells, the method comprising: obtaining a first sample from the subject, the first sample including the target cells; characterizing cell membrane order in the target cells of the first sample using the method as defined in claim 1; treating the patient with the treatment; obtaining a second sample from the subject after the treatment, the second sample including the target cells; characterizing cell membrane order in the target cells of the second sample using the method as defined in claim 1; assessing the effect of the treatment by comparing the cell membrane order in the target cells of the first and second samples.

Advantageously, the proposed method allows assessment of the effect of the treatment on the target cell relatively easily.

For example, the treatment is an anticoagulant treatment and the target cells are platelets, the effect of the treatment being detectable through an increase in the cell membrane order in the platelets. In another example, the treatment includes administering to the patient a statin or clopidogrel.

In another broad aspect, the invention provides a method for characterizing order in a lipid membrane, the method comprising staining the lipid membrane with di-4-ANEPPDHQ to produce a stained membrane; irradiating the stained cell with an excitation light, the excitation light being capable of inducing fluorescence in the di-4-ANEPPDHQ; measuring a fluorescence spectrum of the stained membrane; characterizing the order in the lipid membrane order by computing a spectral signature of the stained membrane from the fluorescence spectrum, the spectral signature providing a character the order in the lipid membrane. Therefore, this proposed method is also capable of characterizing lipid membranes other than cell membrane, such as, for example, the lipid membrane of a microparticle, the lipid membrane of a yeast, the lipid membrane of a bacteria, an artificial lipid membrane and the lipid membrane of a virus.

In another broad aspect, the invention provides a method for characterizing cell membrane order in a cell, the method comprising: staining the cell with a membrane order stain to produce a stained cell; irradiating the stained cell with an excitation light, the excitation light being capable of inducing fluorescence in the membrane order stain; measuring a fluorescence spectrum of the stained cell; and characterizing the cell membrane order by computing a spectral signature of the stained cell from the fluorescence spectrum, the spectral signature providing a character of the cell membrane order.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1: illustrates that the Di-4-ANEPPDHQ dye is excited at 488 nm and emits fluorescence in at least two bands centred on 575 nm and 675 nm, the relative intensity of the light emitted in each band depending on the order of the lipid environment, allowing ratiometric (675/575 nm) measurement (Rf) by flow cytometry;

FIG. 2: illustrates that reproducible ratiometric Di-4-ANEPPDHQ staining can be achieved in diluted plasma;

FIG. 3: illustrates the concordance of fluorescence ratios of the Di-4-ANEPPDHQ dye calculated by two different techniques (confocal fluorescent microscopy and flow cytometry) following cholesterol depletion and cholesterol enrichment;

FIG. 4: illustrates the time evolution of the effects of cholesterol depletion determined using Di-4-ANEPPDHQ staining analysis by flow cytometry;

FIG. 5: illustrates that the Di-4-ANEPPDHQ staining reflects alterations of the lipid bilayer order obtained with methods other than cholesterol depletion, and more specifically by cholesterol clustering (filipin) or cholesterol hydrolysis (cholesterol oxydase);

FIG. 6: illustrates that the Di-4-ANEPPDHQ staining coupled with flow cytometry analysis allows the detection of alterations of the lipid bilayer obtained after platelet activation;

FIG. 7: illustrates that Di-4-ANEPPDHQ staining of platelets correlates with platelet function in patients with a stable coronary artery disease;

FIG. 8 illustrates that the Di-4-ANEPPDHQ staining allows for the detection of a platelet subpopulation in conditions of high shear stress;

FIG. 9: illustrates that Di-4-ANEPPDHQ staining detects modifications of platelet membrane lipid ordered phase induced by statin treatment;

FIG. 10: illustrates that Di-4-ANEPPDHQ staining allows for the detection and quantification of cell apoptosis by flow cytometry;

FIG. 11: illustrates that Di-4-ANEPPDHQ staining allows for the detection of subtypes of cell microparticles by flow cytometry;

FIG. 12: illustrate that Di-4-ANEPPDHQ staining allows the characterization of the fluorescent spectrum of cells depending on their lipid order obtained with cholesterol and/or sphingomyelin depletion;

FIG. 13 illustrates the fluorescence spectrum of Di-4-ANEPPDHQ;

FIG. 14 illustrate that Di-4-ANEPPDHQ staining allows the characterization of the fluorescent spectrum of cells depending on their lipid order obtained with platelet activation;

FIG. 15 illustrates that Di-4-ANEPPDHQ staining allows the characterization membrane signature of cells depending on their lipid order obtained with platelet activation;

FIG. 16 illustrates that Di-4-ANEPPDHQ staining allows the characterization membrane signature of cells depending on their lipid order obtained with cholesterol and/or sphingomyelin depletion;

FIG. 17 illustrates the kinetics of the Di-4-ANEPPDHQ staining measured as described in FIG. 10 in comparison with kinetics of various detection method of apoptosis;

FIG. 18 illustrates that Di-4-ANEPPDHQ staining is correlated to late apoptosis detected by DNA fragmentation and cell permeability measurements; and

FIG. 19 illustrates that the Di-4-ANEPPDHQ staining allows the detection of a platelet subpopulation in patients with coronary artery disease and its regulation with the antiplatelet drug clopidogrel.

### DETAILED DESCRIPTION

The following definitions apply to the terminology used throughout this specification:

Membrane: in this document, the word membrane relates to complete cell membranes and to portions of single layers and dual layers of lipids in eukaryotic cells, procaryotic cells, and viruses.

Fluorescent marker: in this document, a fluorescent marker is a fluorescent molecule which includes a specific affinity, either by itself or when conjugated to an antibody or to any other suitable molecule, for a membrane component and is capable of signaling its presence. Examples of such fluorescent markers include filipin, annexin V, PSS-380, MC540 (merocyanin), and a conjugate of the choleric toxin.

Fluorescent sensor: in this document, a fluorescent sensor is a molecule which absorbs light at a specific wavelength (excitation wavelength), and which, by fluorescence, emits light at a higher wavelength (Stockes' law). The emission wavelength is a function of the degree of order of the membrane surrounding the sensor. In opposition to the fluorescent marker, the fluorescent sensor is inserted into the membrane because it has an affinity for lipids. Examples of such fluorescent sensors include di-4-ANEPPDHQ, laurdan and prodan.

Membrane fluidity: the fluidity of the membrane is defined as the mobility of lipids in a plane defined by the membrane.

Membrane order: the membrane order represents the arrangement of the lipid chains in the membrane [1]. It can be defined as the state of arrangement of membrane lipids, proteins and sugars. This notion defines globally two types of structures: ordered liquid structures and disordered liquid structures. These structures present various degrees of fluidity. [2, 3]

Di-4-ANEPPDHQ: This compound is 1-[2-Hydroxy-3-(N,N-di-methyl-N-hydroxyethyl)ammoniopropyl]-4-[β-[2-(di-n-butylamino)-6-napthyl]vinyl]pyridinium dibromide, also known as JPW5029. This terminology also describes derivatives of the above-detailed compound that have similar membrane order sensitivity.

The principle of detection described herein is based on the variations in the emission wavelengths of a fluorescent sensor according to the order of the surrounding membrane [41, 42]. This method allows a direct measure of the membrane order of a cell.

This type of staining is different from staining based on the asymmetric membrane expression of phosphatidyl serine measured by the binding of annexin V or of the PSS-380[43], a change of intensity of fluorescence induced by a change of membrane potential as described with the other types of dyes of which the F2N12S [44, 45], or a modification of the composition or the density of lipids of the membrane (MC540).

Many compounds are currently in use to characterize membranes. A few examples are given hereinbelow, along with the manner in which the proposed method distinguishes itself over these examples.

Some compounds, such as annexin V and derived compounds specifically targeting the phosphatidylserine, such as the PSS-380, are staining specifically the externalisation of the phosphatidylserine; their binding does not depend on the degree of order of the membrane. These types of compound only measure the appearance of a lipid to the cell surface and not the global order of the membrane as the Di-4-ANEPPDHQ does.

The compounds MC540 and merocyanine are markers of “lipid packing” in the membrane. Moreover, Waczulikova and al. [46] showed that these compounds' binding is strongly dependent on the contents in phosphatidylserine. According to Wilson-Ashworth and al., MC540 is not thus comparable to Laurdan, which is a non specific dye inserted into the membrane and sensitive to the degree of order [1]. The same type of comparison can be made with di-4-ANEPPDHQ which was described as being sensitive to the membrane order and which is non specifically inserted into the membrane bilayer [41, 42].

1,6-diphenyl-1,3,5-hexatriene (DPH) is a UV excitable dye, as opposed to di-4-ANEPPDHQ, which is excitable with visible light. DPH is not specifically bound in the membrane and allows measurements of the anisotropy of the emitted light. Methods using DPH measure modifications in the polarization of the light emitted after excitement by a source of polarized light. This principle of measurement thus requires the use of specialized equipment and does not allow, in opposition to the proposed method, the analysis using a conventional flow cytometer [47]. Moreover, the measurement of anisotropy allows an evaluation of the fluidity of the membrane and not directly of the membrane order [47]. Finally, the polarization of the light emitted by the 1,6-diphenyl-1,3,5-hexatriene is dependent in fact on its interaction with proteins in the membrane [48].

Bis-pyrene is a sensor inserted into the membrane bilayer into the membrane formed by two pyrene nuclei. The monomer of bis-pyrene emits between 370 and 400 nm. In a less fluid environment, the interactions between monomers increase, allowing the formation of excimers emitting at 480 nm. This dye allows the measure of the fluidity of the membrane in the same way as the DPH, it is thus different from di-4-ANEPPDHQ which is directly sensitive to the membrane order. Furthermore, bis-pyrene is excitable by UV light and emits in the UV region of the spectrum, or in the near visible light region. Finally the bis-pyrene is not useful in cells, as its binding is very weak in cellular membranes [1].

The hydroxy-flavone compound F2N12S is described by some authors as sensitive to an increase of the negative charge of the cell surface due to the externalization of the phosphatidylserine, to a modification of the membrane polarity and to the degree of hydratation of the membrane. These characteristics are thus different from the properties of the di-4-ANEPPDHQ. Moreover, the excitation wavelength of this compound is 407 nm, which is different to that of the di-4-ANEPPDHQ and not applicable with common flow cytometers.

The proposed method, by using sensors such as di-4-ANEPPDHQ or Laurdan, is the only one which allows measurements in real time of the membrane order in living cells. This method is based on the detection of the modifications of the emission spectrum and/or the intensity of the emission by fluorescent sensors sensitive to the membrane order in the area surrounding the sensor.

Other examples of suitable fluorescent sensors that could be used include dyes of the family or derived from the styryl group, such as the di-4-ANEPPDHQ, or from the naphthalene group, such as Laurdan or Prodan.

Excitation of the sensor is provided by a light source (for example a laser) and determination of the intensity of the fluorescence emitted by the sensor associated to a specific membrane order is measured by any method of measurement of the intensity of fluorescence or of determination of the emission spectrum (for example by flow cytometry (fluorescence-activated cell sorting (FACS)) or by spectrofluorimetry, among other possibilities).

Quantification of the membrane order in cells can be performed by measuring the area under the curve (AUC) of whole fluorescence spectra, by calculating a ratio (Rf) between total intensities contained in bands in the fluorescence spectra peaks of fluorescence of the emission spectrum, or by any other evaluation based on the modification of the emission spectrum of the sensor. This quantification allows quantifying the global membrane order of a part of a cell or a microorganism. The calculation of the generalized polarization (Gp) can be also used as criterion of differential analysis of the intensities of peaks 1 (Ipic1) and peaks 2 (Ipic2).

$$Rf = \frac{Ipic 1}{Ipic 2} Gp = \frac{Ipic 1 - Ipic 2}{Ipic 1 + Ipic 2}$$

Also, as detailed hereinbelow, in some embodiments of the invention, a signature of the fluorescence spectra including fluorescence intensity information measured over more than 2 spectral bands is computed to obtain a characteristics of the cells stained with the fluorescent sensor.

### Example 1

### Staining Method of Blood Platelets or Isolated Cells with Di-4-ANEPPDHQ

After isolation, blood platelets or cells have been resuspended in a saline buffer (calcium free Tyrode buffer (TBS) or Phosphate buffer (PBS), respectively). Blood platelets and cells are resuspended at the respective final concentrations of 200000/μL and 530000/ml. Working solution of di-4-ANEPPDHQ (Invitrogen) is prepared from the ethanolic stock solution (1.5 mM) by dilution in PBS or TBS to achieve a concentration of 100 μM.

The sensor (di-4-ANEPPDHQ) is mixed to a final concentration of 10 μM with platelets (20000/μL final) and cells (480000/μL). When necessary, TBS or PBS are used for dilutions. The final volume is 50 μL.

After incubation for 5 min at room temperature (20-25° C.), this 50 μL volume is diluted with 250 μL of TBS or PBS and analyzed by flow cytometry.

While the above mentioned parameters for incubation have been used predominantly in the examples described hereinbelow, other parameters are also within the scope of the present invention, such as the parameters mentioned in the summary of the invention section of the present document.

### Example 2

### Direct Staining Method of Blood Platelets from Whole Blood with the Di-4-ANEPPDHQ

The blood is sampled in a conventional manner using 0.105M sodium citrate or heparin or EDTA (Diaminoethanetetraacetic acid) or antithrombins such as PPACK (Phe-Pro-Arg-chloromethylketone) and is centrifuged at 120 g for 15 minutes.

The supernatant is the Platelet Rich Plasma (PRP) and typically contains about 200000 to about 300000 platelets/μL. The PRP is separated and diluted 20 times in calcium free TBS, thereby obtaining a diluted PRP. Afterwards, the diluted PRP (75 mL) is mixed with 25 μL of the working solution of di-4-ANEPPDHQ and left to incubate 5 min at room temperature (20-25° C.). Finally, the incubated mixture is diluted with 250 μL of TBS or PBS and analysed by flow cytometry.

### Example 3

### Analysis and Determination of the Ratio of Fluorescence (Rf) by Flow Cytometry Using Di-4-ANEPPDHQ

Blood platelets and cells are detected and identified according to their forward scatter (FSC) and size scatter (SSC). A total of 5000-10000 particles are typically analysed. The excitation light is provided by a 488 nm laser, but could be achieved with lasers having wavelengths between 400 and 500 nm, among other possibilities. The emission fluorescence associated with liquid ordered domains is acquired between 575+/−25 nm, but the bandwidth could be narrower or wider, for example contained between 500 and 600 nm. The emission fluorescence associated with liquid disordered domains is acquired between 675+/−25 nm, but the bandwidth could be narrower or wider, contained for example between 650 and 750 nm.

Amplifications and gains associated with both fluorescences are adjusted on calibrated fluorescent beads or on a control cell population, to obtain a ratio of fluorescence and to allow increases or decreases of the fluorescences and of the ratio. The ratio of fluorescence (Rf) can be calculated in several ways:

1—Determination by flow cytometry of the ratio of fluorescence of each particle/cell analysed and calculation of the mean or median of the Rf of the selected population of cells.

2—Determination of the mean or median of the two fluorescences described earlier for the selected population by flow cytometry and calculation of the ratio of fluorescence (Rf).

3—Computation of Rf from the area under the curve for the studied population.

### Example 4

### Analysis and Determination of the Percentage of Apoptotic Cells in a Population

Cells are detected, identified and segregated according to their size using forward scatter (FSC) and granularity (SSC). A total of 5000-10000 particles are typically analyzed. The excitation light is provided by a 488 nm laser, but could be achieved with lasers having wavelengths between 400 and 500 nm, among other possibilities. The emission fluorescence associated with liquid ordered domains is acquired between 575+/−25 nm, but the bandwidth could be narrower or wider, for example contained between 500 and 600 nm. The emission fluorescence associated with liquid disordered domains is acquired between 675+/−25 nm, but the bandwidth could be narrower or wider, contained for example between 650 and 750 nm.

Amplifications and gains associated with both fluorescences are adjusted on calibrated fluorescent beads or on a control cell population to obtain a ratio of fluorescence and to allow increases or decreases of the fluorescences and of the ratio. The ratio of fluorescence (Rf) is determined for each particle analysed by computing the ratio between the fluorescence intensity at 675 nm and the fluorescence intensity at 575 nm. By plotting FSC or SSC as a function of Rf for all particles on a logarithmic scale graph, the percentage of apoptotic cells can be determined as being the percentage of cells in the studied population having a Rf higher than a control population.

### Example 5

### Di-4-ANEPPDHQ Dye is Excited at 488 nm and Emits Fluorescence at 575 nm and 675 nm Depending on the Order of the Lipid Environment Allowing Ratiometric (675/575 nm) Measurement (Rf) by Flow Cytometry

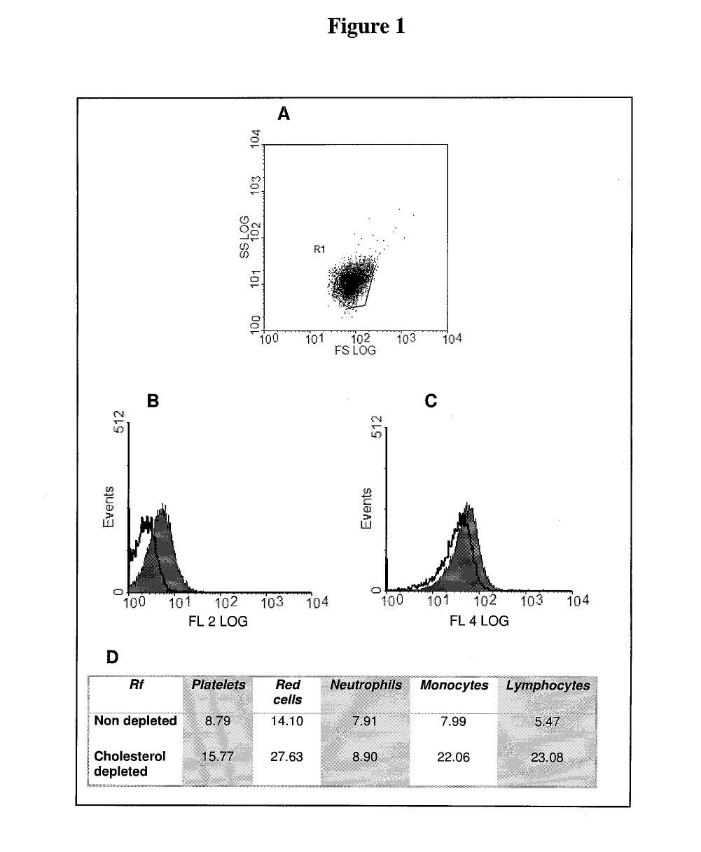

Referring to FIG. 1, di-4-ANEPPDHQ fluorescence was measured on purified washed platelets prepared according to the protocol mentioned hereinabove (Panel A). The emitted median fluorescence intensity (MFI) was measured at wavelengths of 575±25 nm (FL2 LOG on Panel B) and of 675±25 nm (FL4 LOG on Panel C) corresponding respectively to the more ordered and less ordered membranes on an EPICS XL flow cytometer (Beckman Coulter). After cholesterol depletion with 10 mM Methyl-β-cyclodextrin (37° C., 30 min) and staining with 10 μM of di-4-ANEPPDHQ, the fluorescence intensity shifted to lower values (empty curve) compared with control undepleted platelets (filled curve). The ratio of fluorescence (Rf) between the FL4 LOG MFI and FL2 LOG MFI can be calculated from the median fluorescence intensities in platelet, red cells and leucocytes (Panel D). Therefore, this example shows that it is possible to characterize cell membrane order in bulk samples. Also, as illustrated in panel D, different cell types are influenced differently by cholesterol depletion.

### Example 6

### Reproducible Ratiometric Di-4-ANEPPDHQ Staining can be Achieved in Diluted Plasma

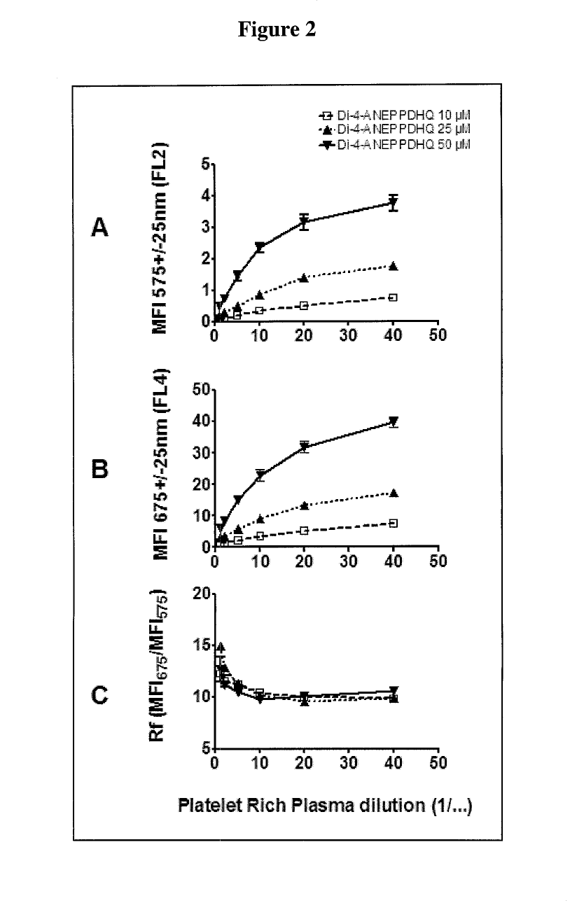

Referring to FIG. 2, increasing concentrations of di-4-ANEPPDHQ (10 to 50 μM) were used to stain platelets with various concentrations of plasma achieved by diluting the Platelet Rich Plasma obtained by centrifugation of the whole blood with Tyrode Buffer (TBS), as detailed hereinabove. The fluorescence intensity of the di-4-ANEPPDHQ increased in the presence of low concentration of plasma and further again when concentration of di-4-ANEPPDHQ were higher (see Panels A and B). The ratio of fluorescence varied at low plasma dilution but was stable at dilution 1/10 and higher (Panel C) (N=2).

### Example 7

### Concordance of Fluorescence Ratios of the Di-4-ANEPPDHQ Dye Calculated by Two Different Techniques (Confocal Fluorescent Microscopy and Flow Cytometry) Following Cholesterol Depletion and Cholesterol Enrichment

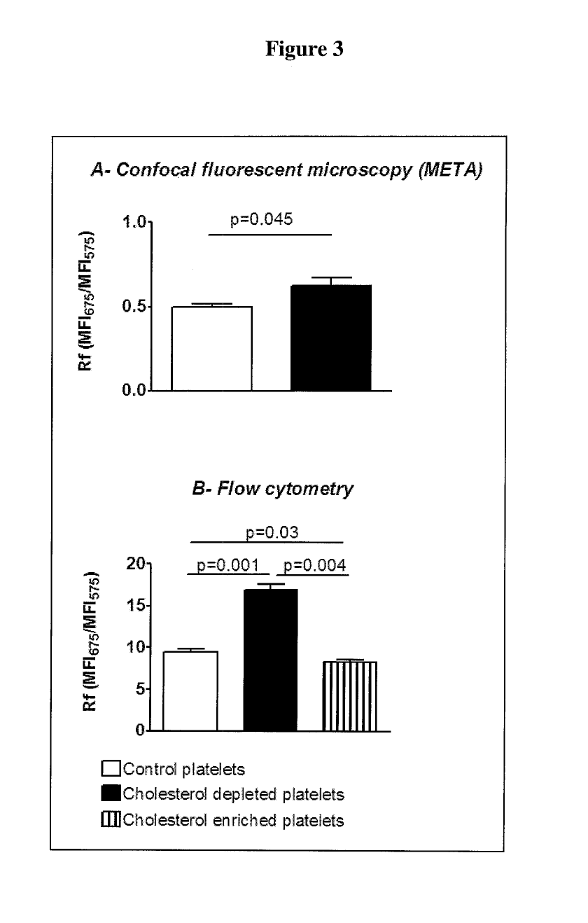

Referring to FIG. 3, fluorescence intensities were measured by confocal fluorescent microscopy and flow cytometry on control platelets, cholesterol depleted platelets (10 mM Methyl-β-cyclodextrin, 37° C., 30 min) and cholesterol enriched platelets (2 mM Cholesterol-MCD complex, 37° C., 30 min).

A META confocal fluorescence microscope system (Zeiss, Toronto) was used to measure the intensities of fluorescence between 534 to 599 nm and between 650 to 684 nm allowing calculating the fluorescence ratio (Rf) prepared according as described hereinabove. Consistently with previous published data on neutrophils (Jin L et al. Biophys J; 2006), the Rf was higher after depletion compared to control platelets (measures on at least 5 platelets of four different donors; p=0.045) (Panel A).

Similarly to these results, the fluorescence ratio (Rf) calculated from flow cytometry data is increased after cholesterol depletion and decreased after cholesterol enrichment (Panel B; N=5).

### Example 8

### Time Evolution of the Effects of Cholesterol Depletion Determined Using the Di-4-ANEPPDHQ Staining Analysis by Flow Cytometry

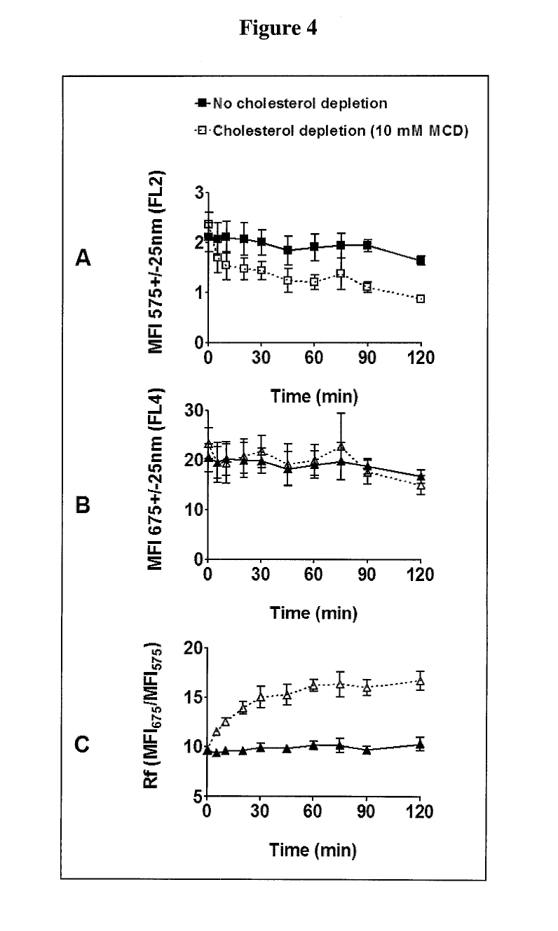

FIG. 4 illustrates medians of fluorescence intensities for FL2 at 575 nm (Panel A) and for FL4 at 675 nm (Panel B) in platelets during the cholesterol depletion (10 mM of Methyl-β-cyclodextrin, room temperature) and after staining with 10 μM of Di-4-ANEPPDHQ. The fluorescence associated to the more ordered membrane (575 nm) decreased whereas the fluorescence emitted at 675 nm remained stable. The corresponding ratio of fluorescence (Rf) between the emission intensities at 575 nm and 675 nm increased during the incubation. (Panel C) (N=3). Substantially stable fluorescence ratio measurement can be achieved after 30 minutes of cholesterol depletion.

### Example 9

### The Di-4-ANEPPDHQ Staining Reflects Alterations of the Lipid Bilayer Order Obtained with Methods Other than Cholesterol Depletion

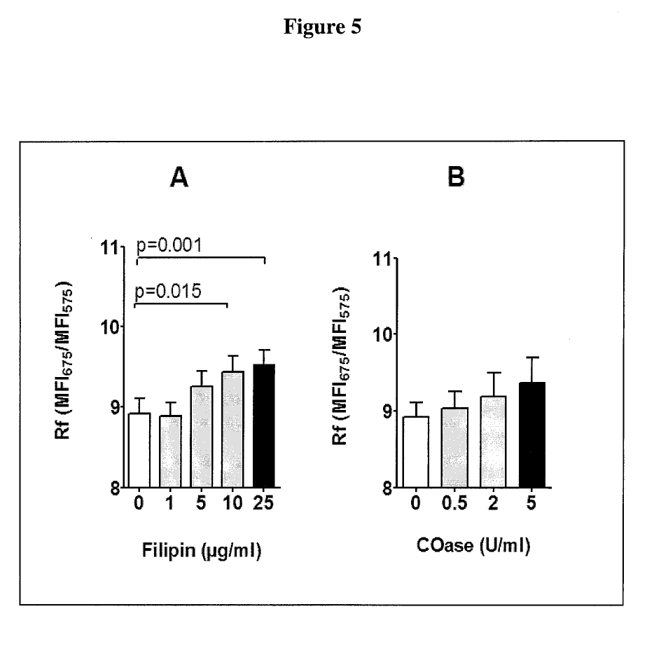

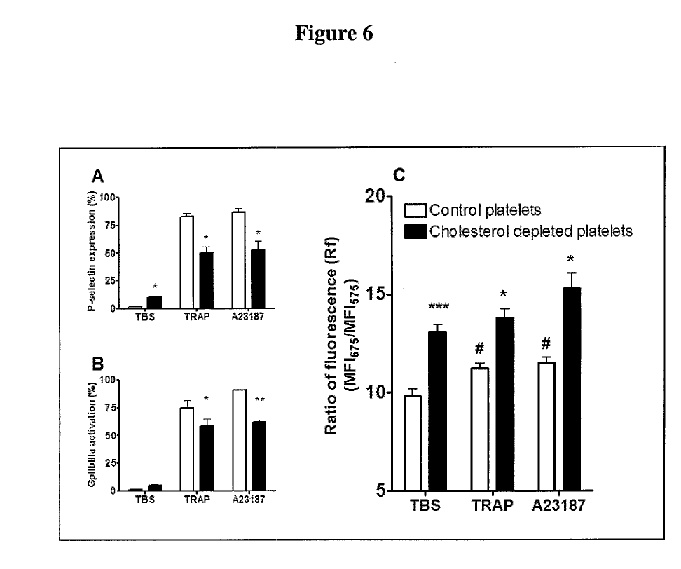

The antibiotic filipin is known to cluster membrane rafts without changing the cholesterol content of the cell. FIG. 5 illustrates the filipin dose-dependency of Rf measured by flow cytometry after incubation with platelets for 30 minutes at 37° C. and different concentrations of filipin. (Panel A) (N=5) Similarly, the treatment of platelets with increasing concentrations of cholesterol oxydase (COase) which transforms the cholesterol into cholestenone tends to increase the ratio. (FIG. 6B) (N=5)

### Example 10

### The Di-4-ANEPPDHQ Staining Coupled with Flow Cytometry Analysis Allows for the Detection of Alterations of the Lipid Bilayer Obtained after Platelet Activation

FIG. 6 illustrates that the cholesterol depletion of washed platelets associated with incubation with 10 mM of Methyl-β-cyclodextrin for 30 minutes at room temperature decreased the expression of platelet activation marker P-selectin (Panel A) and of activation of the activated GpIIbIIIa receptors (Panel B), after addition of 10 μM of the thrombin receptor agonist peptide-6 (TRAP) or 50 μM of the calcium ionophore A23187. In the same conditions, the Rf was significantly increased by both agonists (Panel C; #: p<0.05 vs. non activated platelets incubated with TBS) and the methyl-β-cyclodextrin increased the Rf (* p<0.05; *** p<0.001 vs. control platelets).

Thus, platelet activation induces membrane modifications that are detectable by flow cytometry analysis with di-4-ANEPPDHQ staining and characterized by an increase of the fluorescence ratio.

### Example 11

### Di-4-ANEPPDHQ Staining of Platelets Correlates with Platelet Function of Patients with a Stable Coronary Artery Disease

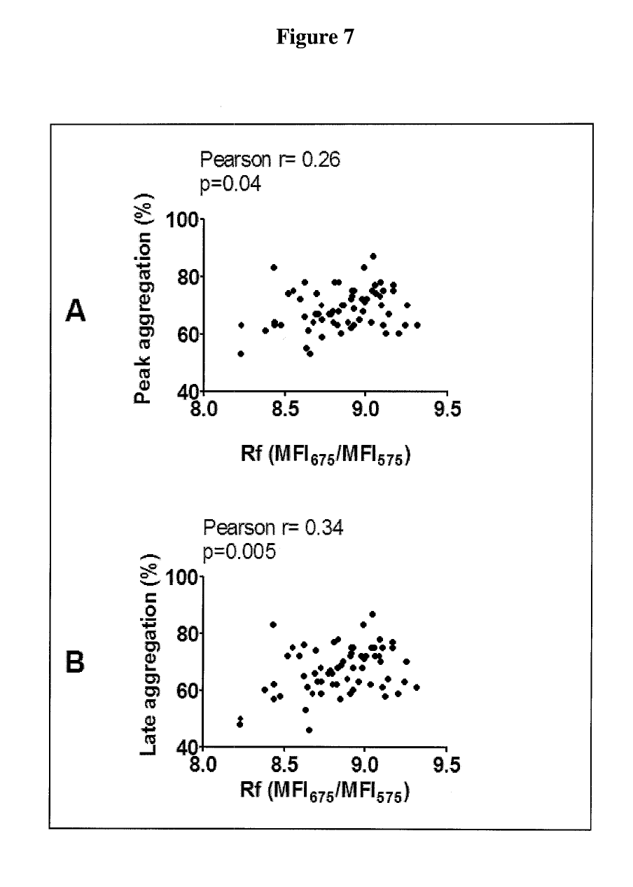

Platelets from patients with coronary artery diseases were both isolated in Tyrode Buffer and stained with Di-4-ANEPPDHQ, and assessed by platelet aggregation measured in platelet rich plasma. As seen in FIG. 7, maximum aggregation (Panel A) and aggregation at 6 minutes (Panel B) after the addition of 20 μM Adenosine 5′ diphosphate (ADP) were measured (N=65). Coefficient of correlations were calculated using the Pearson's correlation test. Di-4-ANEPPDHQ ratio of fluorescence calculated from flow cytometry data significantly correlated with platelet response to the physiological agonist ADP.

### Example 12

### Di-4-ANEPPDHQ Allows Detection of Modifications of Platelet Membrane Lipid Ordered Phase Following the Administration of the Antiplatelet Drug Clopidogrel

Clopidogrel is an antiplatelet drug which active metabolite directly blocks the purinergic P2Y12 receptor. This metabolite possesses a free thiol activity and has recently been shown to depolymerise the P2Y12 receptor participating in the removal of the receptor from lipid rafts (ordered phase) (Savi P et al. PNAS; 2006).

A PREPAIR randomized trial compared 3 different clopidogrel regimens used before coronary angiography and planned percutaneous intervention in patients with a stable coronary disease. Groups were defined as follow: Group A, clopidogrel 300 mg the day before (≧15 hours)+75 mg the morning of the interventional procedure; Group B, clopidogrel 600 mg the morning of (2 hours before) the interventional procedure; Group C, clopidogrel 600 mg the day before (≧15 hours) and 600 mg the morning of 2 hours before) the interventional procedure. All analyses were performed blinded to treatment allocation. After administration of clopidogrel, Rf is significantly decreased in groups A (8.80+/−0.2 vs. 8.68+/−0.22; p<0.001) and B (8.90+/−0.24 vs. 8.77+/−0.27; p=0.006), but not in group C (8.69+/−0.29 vs. 8.69+/−0.21; p=0.88), all results being mean+/−strandard deviation. Thus, the ratio of fluorescence (Rf) of the di-4-ANEPPDHQ staining is decreased by the clopidogrel by reducing the basal in vivo platelet activation state or by acting directly on its receptor P2Y12, located in the more ordered lipid phase.

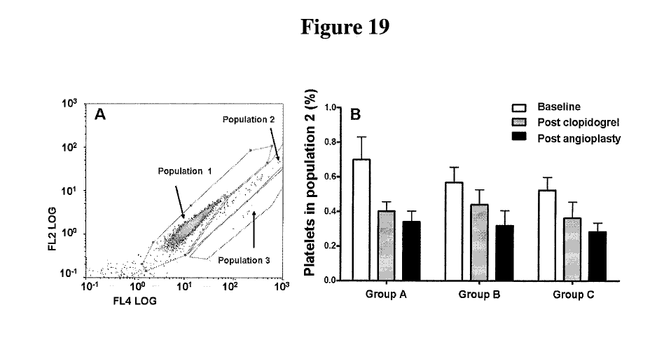

Plotting FL2 as a function of FL4 with no administration of clopidogrel allowed for the identification of sub-populations in samples. For example, one of these sub-populations clustered around Rf of 16.9+/−0.09 as compared to Rf=208.8+/−0.03 for the majority of the platelets. This sub-population represented about 0.46% of platelets before treatment (FIG. 19, panel A, Population 2) and was inhibited in all three patient groups (FIG. 19 panel B). This inhibition is maintained by angioplasty. A multivariate analysis showed a significant influence of clopidogrel treatment (p=0.0009) and pathology severity (p=0.024) on the proportion of cells in population 2.

### Example 13

### Di-4-ANEPPDHQ Staining Detects Modifications of Platelet Membrane Lipid Ordered Phase Induced by Statin Treatment

The statins are HMCoA reductase inhibitors that inhibit the endogenous synthesis of the cholesterol increasing receptor activity to remove cholesterol from blood, thereby protecting against atherosclerosis. Beyond these properties, statins possess pleiotropic effects on inflammation, cell and platelet function. The mechanism for these presumably lipid independent properties is not fully elucidated.

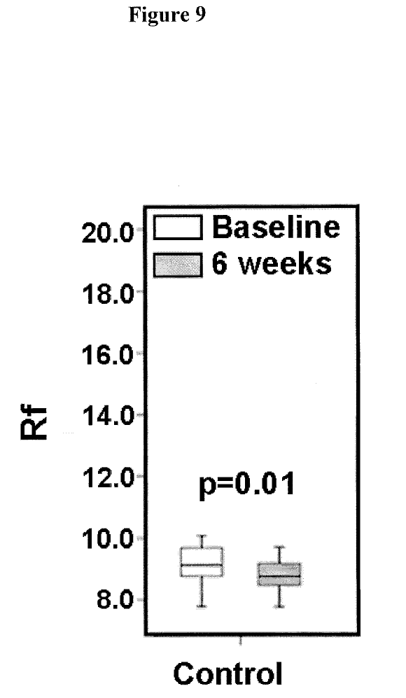

In an open labeled study 20 hyperlipidemic patients were studied before (Baseline) and after a 6-week period of treatment with atorvastatin 40 mg. As seen in FIG. 9, the statin modified di-4-ANEPPDHQ staining (Rf) (p=0.01).

Treatment with atorvastatin normalized the total cholesterol, LDL, and triglyceride plasma levels but unexpectedly increased MLO in platelets (Rf: 9.2+/−0.1 vs. 8.8+/−0.1; p=0.01) and tends to decrease the effects of the cholesterol depletion on the MLO (12.8+/−0.5 vs. 11.7+/−0.2; p=0.09). This change was not correlated to the plasma lipid levels suggesting a distinct mechanism.

The use of the di-4-ANEPPDHQ ratio of fluorescence (Rf) is therefore a useful research tool to fully understand the membrane modifications induced by the statin treatment that could reflect the cellular effects of statins and contribute the understanding of their pleiotropic effects.

### Example 14

### Di-4-ANEPPDHQ Staining Allows the Detection and Quantification of Cell Apoptosis by Flow Cytometry

Apoptosis is a cellular process implicated in numerous physiological and pathological events. Detecting and quantifying apoptosis is of major interest in medicine and biology. Alterations of membranes are among the first events occurring in cell apoptosis, and are widely studied.

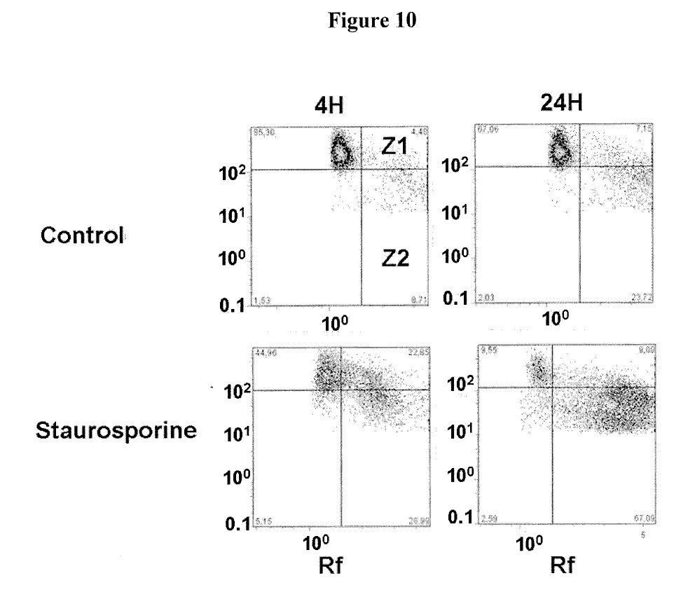

In FIG. 10, two zones (Z1 and Z2) corresponding respectively to cells with increased Rf but no change in size and to cells with high Rf but with decrease in size can be identified. After induction of apoptosis, Rf in cells from zone 2 is more than doubled with respect to the initial population (1.2+/−0.02 vs. 3.2+/−0.2; p<0.001).

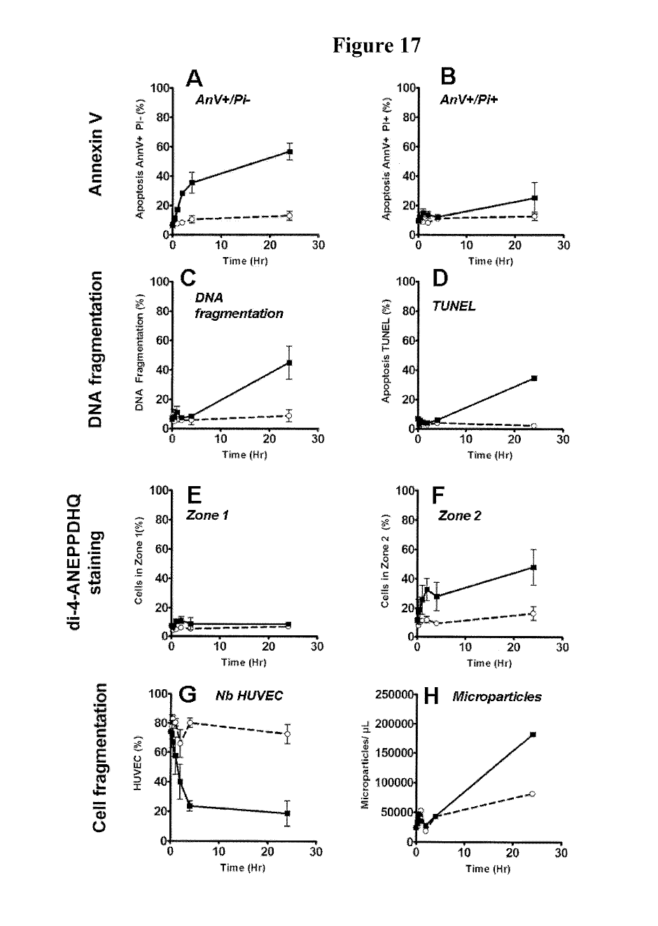

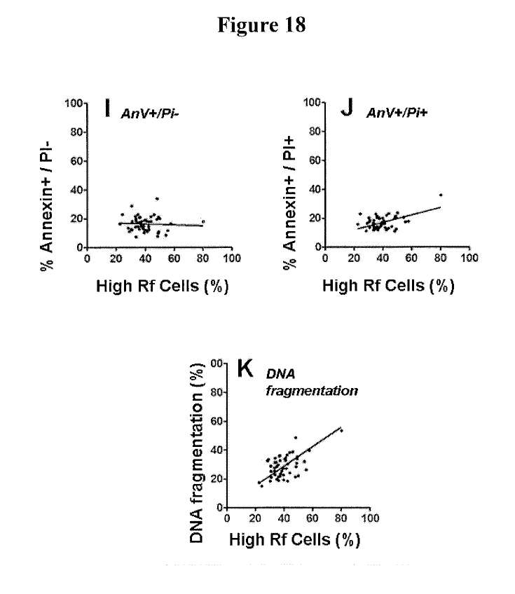

FIG. 17 shows in panels A to D the kinetics of various measures of apoptosis after the addition of staurosoprine through standard methods such as staining with annexin and V-FITC/propium iodine, staining with TUNEL or detection of cells having a reduced DNA content. The proportion of cells in Z1 (panel E) shows practically no time-dependant evolution while cells in zone Z2 (panel F) have a kinetic similar to that of cells experiencing early apoptosis. As seen in panel G, the total proportion of intact cells decreases regularly after induction of apoptosis and the concentration in microparticles is maximal after 24 hrs, as seen in panel H. As shown in panel I of FIG. 18, in patients with acute coronary syndrome or infarction, the percentage of high Rf cells (zones 1 and 2) is not correlated with early apoptosis measurements with annexine+/PI−(r²=0.02; p=0.75), but is correlated with late apoptosis measurements as measured by the percentage of annexine+/PI+cells (panel J; r²=0.21; p=0.001). Also, the percentage of cells with high Rf is significantly correlated with opoptosis measured as the percentage of hypodiploid cells after marking with propidium iodine (panel K; r²=0.31; p<0.001).

### Example 15

### Di-4-ANEPPDHQ Staining Allows the Detection of Subtypes of Cell Microparticles by Flow Cytometry

Microparticles are cellular fragments which are composed of the lipid membrane bilayer of the original cell. It is thus possible to identify microparticles with external markers such as CD42 for platelets. Recent work showed that microparticles are comparable in composition to lipid rafts and that the lipid content could vary upon activation.

Microparticles were obtained from platelets washed and suspended in a buffer comprising Hepes (10 mmol/L), NaCl (137 mmol/L), KCl (5.38 mmol/L), CaCl₂ (5 mmol/L), pH 7.4, after activation for 30 nm at 37° C. by 10 μM of A23187 or by a mixture of convulxin and thrombin (500 ng/mL/0.5 U/mL). After centrifugation for 5 min at 7,200 g, the surnatant including the microparticles was stained with 10 μM di-4-ANEPPDHQ or using annexin V-FITC at 1/20 (v/v) (Becton Dickinson) for 15 min at 25° C.

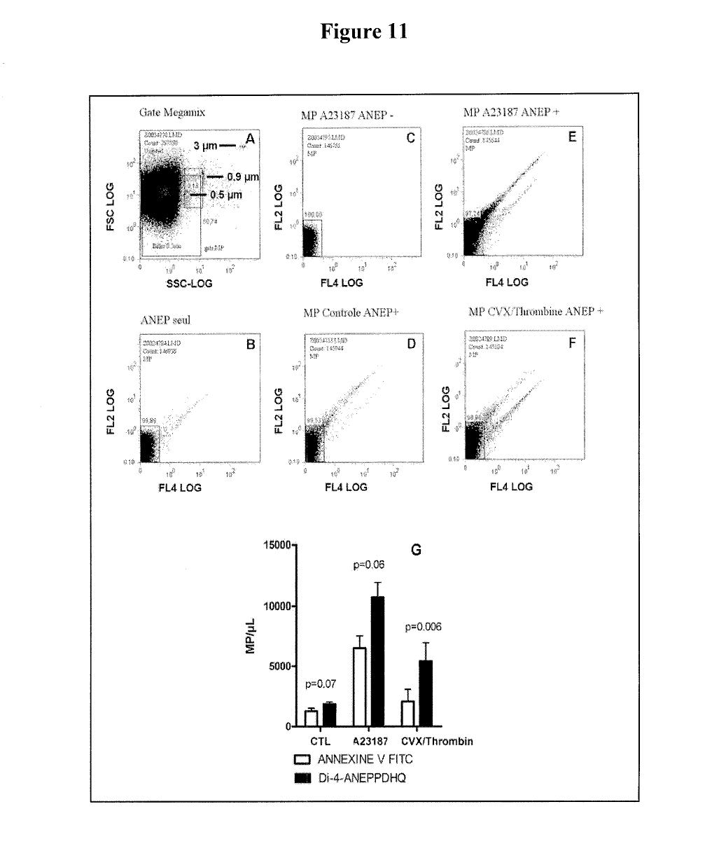

Microparticles were detected by selecting particles included in the MP zone represented in FIG. 11, panel A. The buffer presented only low background noise and no particles were detected in the buffer in absence of di-4-ANEPPDHQ. Two distinct microparticle types were observed in control samples (FIG. 11, panel B). After activation with A23187 (panel E), or mixing with convulxin/thrombin (panel F), more microparticles in both populations were observed with di-4-ANEPPDHQ using the herein above mentioned method than with annexin V-FITC (panel G).

### Example 16

### Di-4-ANEPPDHQ Staining Allows the Characterization of the Fluorescent Spectrum of Cells Depending on their Lipid Order and the Subsequent Determination of a Membrane Signature Represented as a Matrix

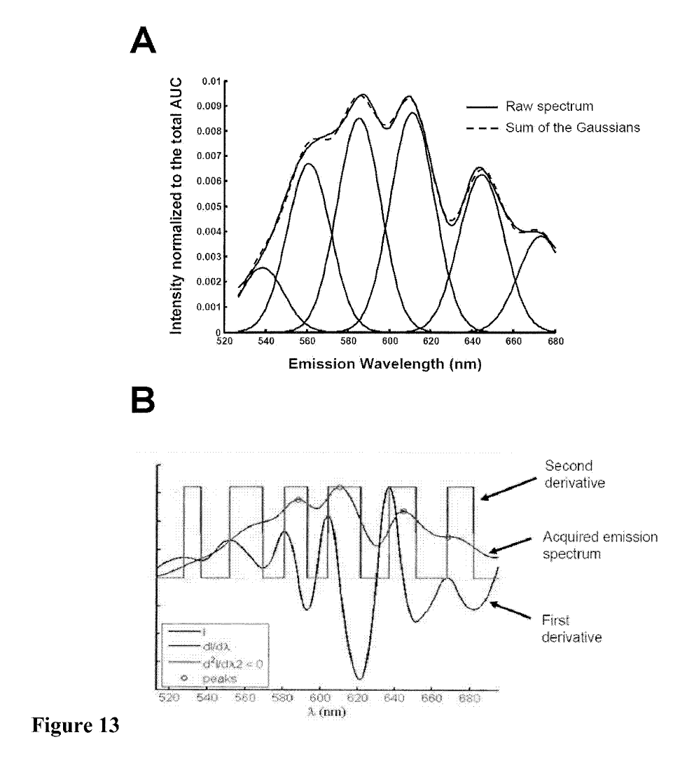

FIG. 13 shows a typical emission spectrum of di-4-ANEPPDHQ acquired using a LSM META confocal microscope with a 488 nm excitation wavelength. Six discrete emission peaks were identified. The acquired emission spectrum was best fitted with the sum of six Gaussians (panel A). The analysis of the acquired emission spectrum led to the determination of four different emission peaks and two shoulders, confirming the gaussian data (panel B). A peak was considered when the first derivative reaches 0 and the second derivative its maximum. Each peak corresponded to distinct liquid order phases. From this spectrum, bands were identified to perform a better characterization of membrane order.

More specifically, FIG. 13 was obtained by suspending blood platelets in TBS in a Poly-D-Lysine covered glass-bottom Petri dish. After washing twice with PBS, adherent platelets were incubated in 10 μM di-4-ANEPPDHQ dissolved in TBS and washed once before spectrum acquisition.

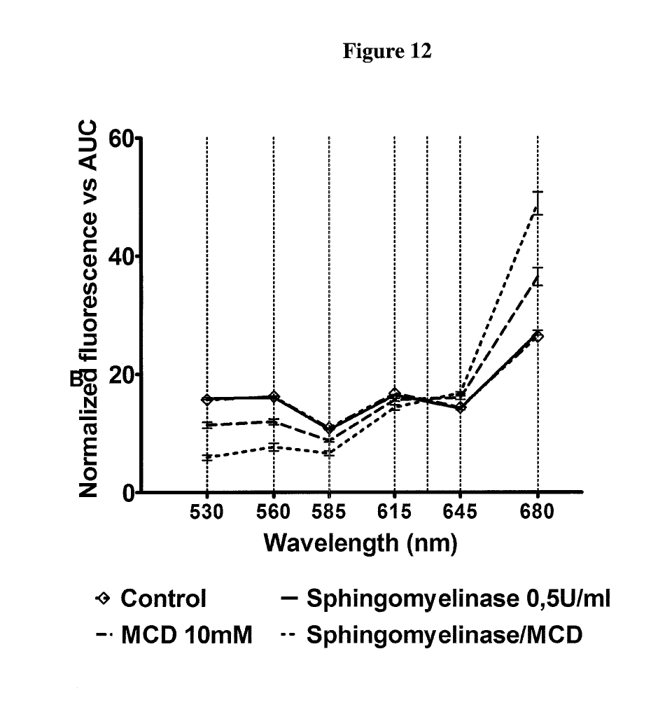

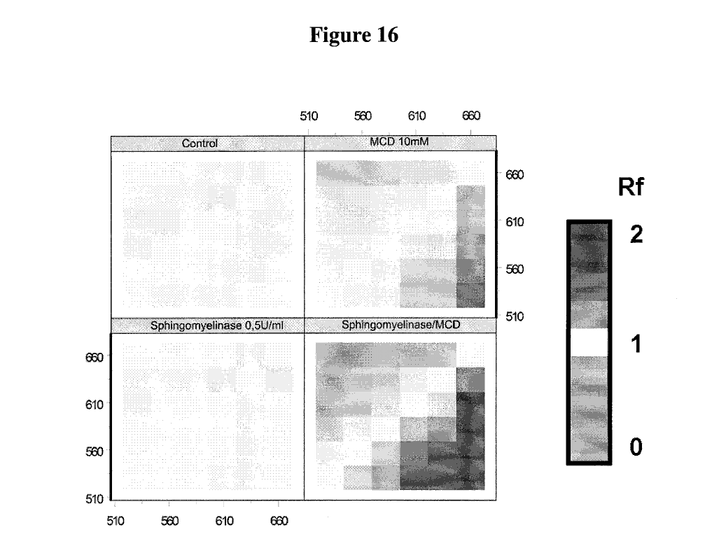

The whole fluorescent emission spectrum of cells (platelets) stained with di-4-ANEPPDHQ can be measured by flow cytometry using 530/30 nm, 560/30 nm, 585/20 nm, 615/30 nm, 645/30 band pass filters and a 660 nm long pass filter, for example. However, other wavelengths are usable in alternative embodiments of the invention. FIG. 12 illustrates the changes of fluorescent intensities measured at each emission wavelength following cholesterol depletion (MCD), sphingomyelinase treatment (SM) or both (SM-MCD) compared to non depleted platelets (CTL). FIG. 16 illustrates that changes in fluorescent intensities measured at the above mentioned wavelengths can be represented as a matrix of ratios of fluorescence intensities. Such a matrix represents the membrane order signature of a cell or of a population of cells with ratios represented as intensity, colors or combination of intensities and colors. Using well-known statistical methods and pattern recognition methods, as well as visual inspection in some embodiments, determination of the order in the membrane is therefore made possible. For example, the measured ratios are compared with ratios obtained using cells having known properties. In an embodiment of the invention, cells infected by a virus are first characterized using the proposed method. Afterwards, diagnosis of infection by the virus is made possible by characterizing cells from a patient and comparing the pattern of fluorescence of the cells of the patient with the pattern of fluorescence of cells infected by the virus.

### Example 17

### Di-4-ANEPPDHQ Staining Allows the Detection and Quantification Effects of a Statin Treatment on Platelets

Statin-induced MLO modifications were quantified in blood platelets from 20 hyperlipidemic patients before and after a 6 weeks treatment with 40 mg atorvastatin.

Membrane signature determination using the herein above described method was achieved by decomposing the di-4-ANEPPDHQ emission spectrum into 6 wavelength band paths corresponding to decreasing MLO: 530/30 nm, 560/20 nm, 585/30 nm, 615/30 nm, 645/30 nm and 660 long path. Spectral FC allowed a rapid and quantitative analysis of the MLO modifications in platelets.

Determination of the di-4-ANEPPDHQ spectrum by spectral FC and confocal microscopy confirmed the red shift of treated platelets. Determination of the spectrum variations induced by the statin treatment for only 4 patients shows a slight although non significant increase of the 530/30 nm band path (14.9+/−0.3 vs. 16.1+/−0.3; p=0.12), corresponding to the most ordered state of the membrane detected by the sensor.

This is the first ratiometric approach for the quantification of MLO in living cells by flow cytometry. This new tool to study cell membrane microdomains is compatible with clinical studies. The increased MLO after a statin treatment in hyperlipidemic patients was not expected since it has been shown that statin decreases the concentration of cholesterol in platelets membranes. However, these results are suggesting that a statin treatment does affect the platelet membrane order by a cholesterol-independent mechanism non-related to lipid plasma concentrations.

### Example 18

### Di-4-ANEPPDHQ Staining Allows Characterization of Platelets Activation

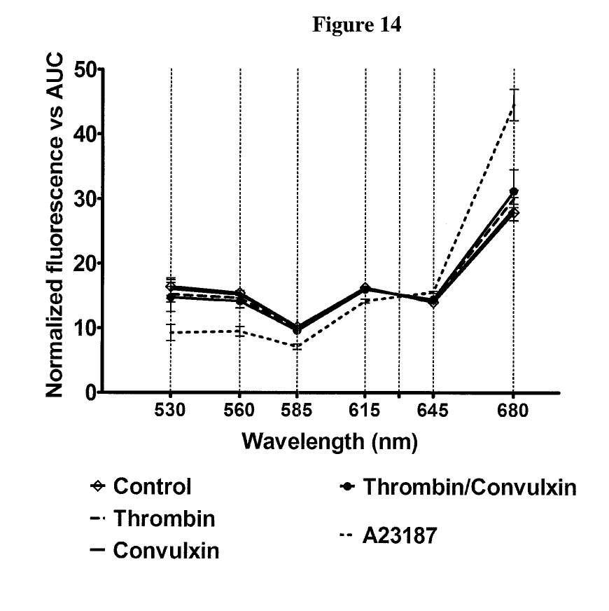

Platelets were suspended in a Calcium Tyrode buffer to a concentration of 20 000/μL and then incubated for 30 minutes at 37 C in presence of 100 μM of ADP (Sigma-Aldrich), 20 μM of TRAP-6 (Bachem, Torrance, Calif.), of 0.5 U/mL of thrombine (Sigma Chemical Co.), of a mixture of thrombin and convulxin (Centerchem Inc, Norwalk, Conn.) at 0.5 U/mL and 500 ng/mL final concentration respectively or of 10 μM calcium ionophore A23187. After incubation, platelets were resuspended and centrifugated. As seen in FIG. 14, each of the substances changes the various fluorescence peaks to a different extent. When compared to controls, thrombine decreased significantly fluorescence between 530 and 585 nm and increased significantly fluorescence at wavelengths larger than 660 nm, illustrating ratios between pairs of fluorescence intensities for the same treatments.

### Example 19

### Di-4-ANEPPDHQ Staining Allows Characterization of Platelets Activation

We performed spectral FC (BD LSRII) by decomposing the di-4-ANEPPDHQ spectrum into 6 wavelength bandpaths corresponding to decreasing MLO: 530/30 nm, 560/20 nm, 585/30 nm, 615/30 nm, 645/30 nm and 660 long path. Spectral FC allowed a rapid and quantitative analysis of the MLO modifications in platelet microdomains. Platelets were stimulated by soluble agonists—ADP 100 μM, TRAP 20 μM, Thrombin 0.5 U/ml, Thrombin 0.5 U/ml/Convulxin 500 ng/ml (Thr/CVX) and A23187 10 μM.

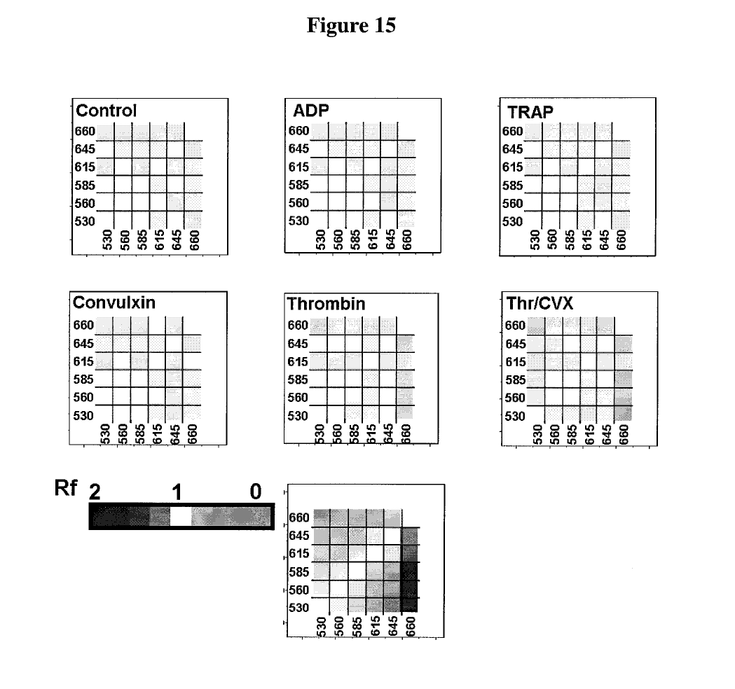

Spectral FC confirmed the decrease of the fluorescence intensities between 515 and 600 nm corresponding to the more ordered phases upon activation by thrombin and A23187 but non significantly for Thr/CVX (FIG. 15). Consistently, the fluorescence intensity of the disordered phase measured with the 660 long path filter was increased.

### Example 20

### Di-4-ANEPPDHQ Staining Allows Detection of a Platelet Sub-Population Generated During Shear Induced Platelet Activation (SIPA)

The Shear Induced Platelet activation (SIPA) experiments were conducted in a Couette-type viscosimeter device to achieve a constant laminar flow rate of 6000 s⁻¹. The platelets, resuspended at a final concentration of 200,000/μL in Tyrode buffer supplemented with 2.5 mM Ca²⁺ were incubated for 15 min at 37° C. with 10 μg/mL von Willebrand factor (vWF) followed by a 5-minute incubation with 10 μM Di-4-ANEPPDHQ (Invitrogen, Burlington, ON) and injected into the viscosimeter, maintained at 37° C. Sub-samples (25 μL) were taken from the suspension at times 0, 30, 60, 120, 270 and 300 sec of shear stress and immediately diluted in 250 μL phosphate buffer for flow cytometry analysis.

The specificity of the SIPA was verified by blocking the binding of vWF to its platelet receptor, GPIb, using the function-blocking monoclonal antibody against GPIb (SZ2, 20 μg/mL. Beckman-Coulter) or the recombinant vWF-binding domain of GPIb, GPG-290 (40 μg/mL, Wyeth, Pharmaceuticals, Madison, N.J.).

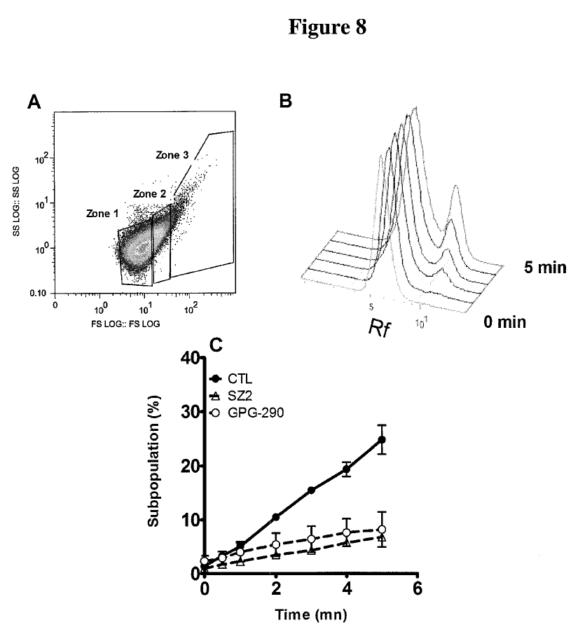

The measures by flow cytometry of the size (FSC) and granularity (SSC) of resting, non aggregated platelets (control sample) allowed to define zones in a FSC/SSC dot plot corresponding to non-aggregated platelets (Zone 1), micro-aggregates involving a small number of platelets (Zone 2) and large aggregates (Zone 3) (FIG. 8, panel A)

Application of a high shear rate (6,000/sec) to platelets led to the generation of a distinct platelet subpopulation in zone 1 with a very high Rf compared to controls (15.9+/−1.2 vs. 8.6+/−0.7; p=0.008) representing 24.7+/−2.6% of the non-aggregated platelets. after 5 min of shear (FIG. 8, panel B). This subpopulation was almost negated when platelets were sheared in the presence of a blocking anti-vWF antibody or GPG-290, a recombinant fragment of the GpIb-IX-V FIG. 20 C.

Pre-incubation of platelets with cytochalasin D or latruncullin B to block actin polymerization also abrogated the generation of this population during the shear treatment.

Visual inspection on confocal microscopy on sheared samples confirmed the apparition of platelets exhibiting a round shape with a di-4-ANEPPDHQ staining shifted to the higher wavelength compared to unsheared platelets.

The present work describes an original approach to detect and monitor platelet activation by measuring the platelet membrane liquid order (MLO). Our results show that the membrane order of platelets is profoundly and differentially altered during activation, depending on the agonist. Moreover, in condition of high shear stress, similar to stenotic arteries, we described the apparition of a unique subpopulation of round-shaped platelets with a highly liquid disordered membrane. This subpopulation presents similarities with previously described highly pro-coagulant platelets and could be of pathological interest in cardiovascular diseases.

Although the present invention has been described hereinabove by way of preferred embodiments thereof, it can be modified, without departing from the spirit and nature of the subject invention as defined in the appended claim

### REFERENCES

1. Wilson-Ashworth, H. A., et al., Differential detection of phospholipid fluidity, order, and spacing by fluorescence spectroscopy of bis-pyrene, prodan, nystatin, and merocyanine 540. Biophys J, 2006. 91(11): p. 4091-101.
2. Binder, W. H., V. Barragan, and F. M. Menger, Domains and rafts in lipid membranes. Angew Chem Int Ed Engl, 2003. 42(47): p. 5802-27.
3. London, E., How principles of domain formation in model membranes may explain ambiguities concerning lipid raft formation in cells. Biochim Biophys Acta, 2005. 1746(3): p. 203-20.
4. Singer, S. J. and G. L. Nicolson, The fluid mosaic model of the structure of cell membranes. Science, 1972. 175(23): p. 720-31.
5. Rukmini, R., et al., Cholesterol organization in membranes at low concentrations: effects of curvature stress and membrane thickness. Biophys J, 2001. 81(4): p. 2122-34.
6. Ritchie, K. and A. Kusumi, Role of the membrane skeleton in creation of microdomains. Subcell Biochem, 2004. 37: p. 233-45.
7. van Lier, M., et al., Adhesive surface determines raft composition in platelets adhered under flow. J Thromb Haemost, 2005. 3(11): p. 2514-25.
8. Pike, L. J., Lipid rafts: bringing order to chaos. J Lipid Res, 2003. 44(4): p. 655-67.
9. Kusube, M., et al., Bilayer phase transitions of N-methylated dioleoylphosphatidylethanolamines under high pressure. Chem Phys Lipids, 2006. 142(1-2): p. 94-102.
10. Kusube, M., H. Matsuki, and S. Kaneshina, Effect of pressure on the Prodan fluorescence in bilayer membranes of phospholipids with varying acyl chain lengths. Colloids Surf B Biointerfaces, 2005. 42(1): p. 79-88.
11. Rajendran, L. and K. Simons, Lipid rafts and membrane dynamics. J Cell Sci, 2005. 118(Pt 6): p. 1099-102.
12. Bodin, S., H. Tronchere, and B. Payrastre, Lipid rafts are critical membrane domains in blood platelet activation processes. Biochim Biophys Acta, 2003. 1610(2): p. 247-57.
13. Hancock, J. F., Lipid rafts: contentious only from simplistic standpoints. Nat Rev Mol Cell Biol, 2006. 7(6): p. 456-62.
14. Lopez, J. A., I. del Conde, and C. N. Shrimpton, Receptors, rafts, and microvesicles in thrombosis and inflammation. J Thromb Haemost, 2005. 3(8): p. 1737-44.
15. Maguy, A., T. E. Hebert, and S. Nattel, Involvement of lipid rafts and caveolae in cardiac ion channel function. Cardiovasc Res, 2006. 69(4): p. 798-807.
16. Insel, P. A., et al., Caveolae and lipid rafts: G protein-coupled receptor signaling microdomains in cardiac myocytes. Ann N Y Acad Sci, 2005. 1047: p. 166-72.
17. Del Conde, I., et al., Tissue-factor-bearing microvesicles arise from lipid rafts and fuse with activated platelets to initiate coagulation. Blood, 2005. 106(5): p. 1604-11.
18. Dietzen, D. J., K. L. Page, and T. A. Tetzloff, Lipid rafts are necessary for tonic inhibition of cellular tissue factor procoagulant activity. Blood, 2004. 103(8): p. 3038-44.
19. Dietzen, D. J., et al., Inhibition of 3-hydroxy-3-methylglutaryl coenzyme A (HMG CoA) reductase blunts factor VIIa/tissue factor and prothrombinase activities via effects on membrane phosphatidylserine. Arterioscler Thromb Vasc Biol, 2007. 27(3): p. 690-6.
20. Mason, R. P., et al., Active metabolite of atorvastatin inhibits membrane cholesterol domain formation by an antioxidant mechanism. J Biol Chem, 2006. 281(14): p. 9337-45.
21. Mason, R. P., M. F. Walter, and R. F. Jacob, Effects of HMG-CoA reductase inhibitors on endothelial function: role of microdomains and oxidative stress. Circulation, 2004. 109(21 Suppl 1): p. 1134-41.
22. Riethmuller, J., et al., Membrane rafts in host-pathogen interactions. Biochim Biophys Acta, 2006. 1758(12): p. 2139-47.
23. Hawkes, D. J. and J. Mak, Lipid membrane; a novel target for viral and bacterial pathogens. Curr Drug Targets, 2006. 7(12): p. 1615-21.
24. Manes, S., G. del Real, and A. C. Martinez, Pathogens: raft hijackers. Nat Rev Immunol, 2003. 3(7): p. 557-68.
25. Taylor, D. R. and N. M. Hooper, The prion protein and lipid rafts. Mol Membr Biol, 2006. 23(1): p. 89-99.
26. Stuermer, C. A., et al., PrPc capping in T cells promotes its association with the lipid raft proteins reggie-1 and reggie-2 and leads to signal transduction. Faseb J, 2004. 18(14): p. 1731-3.
27. Manes, S. and A. Viola, Lipid rafts in lymphocyte activation and migration. Mol Membr Biol, 2006. 23(1): p. 59-69.
28. Gombos, I., et al., Cholesterol and sphingolipids as lipid organizers of the immune cells' plasma membrane: their impact on the functions of MHC molecules, effector T-lymphocytes and T-cell death. Immunol Lett, 2006. 104(1-2): p. 59-69.
29. Razzaq, T. M., et al., Regulation of T-cell receptor signalling by membrane microdomains. Immunology, 2004. 113(4): p. 413-26.
30. Jury, E. C., et al., Atorvastatin restores Lck expression and lipid raft-associated signaling in T cells from patients with systemic lupus erythematosus. J Immunol, 2006. 177(10): p. 7416-22.
31. Brusselmans, K., et al., Squalene synthase: A determinant of raft-associated cholesterol and modulator of cancer cell proliferation. J Biol Chem, 2007.
32. Weerachatyanukul, W., et al., Visualizing the localization of sulfoglycolipids in lipid raft domains in model membranes and sperm membrane extracts. Biochim Biophys Acta, 2007. 1768(2): p. 299-310.
33. Winocour, P. D., et al., Reduced membrane fluidity in platelets from diabetic patients. Diabetes, 1990. 39(2): p. 241-4.
34. Chen, W., et al., Inhibition of cytokine signaling in human retinal endothelial cells through modification of caveolae/lipid rafts by docosahexaenoic acid. Invest Opthalmol V is Sci, 2007. 48(1): p. 18-26.
35. Lusa, S., et al., Depletion of rafts in late endocytic membranes is controlled by NPC1-dependent recycling of cholesterol to the plasma membrane. J Cell Sci, 2001. 114(Pt 10): p. 1893-900.
36. Flores-Borja, F., et al., Altered lipid raft-associated proximal signaling and translocation of CD45 tyrosine phosphatase in B lymphocytes from patients with systemic lupus erythematosus. Arthritis Rheum, 2007. 56(1): p. 291-302.
37. Thaler, C. D., M. Thomas, and J. R. Ramalie, Reorganization of mouse sperm lipid rafts by capacitation. Mol Reprod Dev, 2006. 73(12): p. 1541-9.
38. Biro, E., et al., The phospholipid composition and cholesterol content of platelet-derived microparticles: a comparison with platelet membrane fractions. J Thromb Haemost, 2005. 3(12): p. 2754-63.
39. Schuck, S., et al., Resistance of cell membranes to different detergents. Proc Natl Acad Sci USA, 2003. 100(10): p. 5795-800.
40. Heijnen, H. F., et al., Concentration of rafts in platelet filopodia correlates with recruitment of c-Src and CD63 to these domains. J Thromb Haemost, 2003. 1(6): p. 1161-73.
41. Jin, L., et al., Cholesterol-enriched lipid domains can be visualized by di-4-ANEPPDHQ with linear and nonlinear optics. Biophys J, 2005. 89(1): p. L04-6.
42. Jin, L., et al., Characterization and application of a new optical probe for membrane lipid domains. Biophys J, 2006. 90(7): p. 2563-75.
43. Koulov, A. V., et al., Detection of apoptotic cells using a synthetic fluorescent sensor for membrane surfaces that contain phosphatidylserine. Cell Death Differ, 2003. 10(12): p. 1357-9.
44. M'Baye, G., et al., Fluorescent dyes undergoing intramolecular proton transfer with improved sensitivity to surface charge in lipid bilayers. Photochem Photobiol Sci, 2007. 6(1): p. 71-6.
45. Shynkar, V. V., et al., Fluorescent biomembrane probe for ratiometric detection of apoptosis. J Am Chem Soc, 2007. 129(7): p. 2187-93.
46. Waczulikova, I., et al., Phosphatidylserine content is a more important contributor than transmembrane potential to interactions of merocyanine 540 with lipid bilayers. Biochim Biophys Acta, 2002. 1567(1-2): p. 176-82.
47. Benderitter, M., et al., Simultaneous analysis of radio-induced membrane alteration and cell viability by flow cytometry. Cytometry, 2000. 39(2): p. 151-7.
48. Mely-Goubert, B. and M. H. Freedman, Lipid fluidity and membrane protein monitoring using 1,6-diphenyl-1,3,5-hexatriene. Biochim Biophys Acta, 1980. 601(2): p. 315-27.

1. A method for characterizing cell membrane order in a cell, said method comprising:
staining said cell with di-4-ANEPPDHQ to produce a stained cell;
irradiating said stained cell with an excitation light, said excitation light being capable of inducing fluorescence in said di-4-ANEPPDHQ;
measuring a fluorescence spectrum of said stained cell; and
characterizing said cell membrane order by computing a spectral signature of said stained cell from said fluorescence spectrum, said spectral signature providing a character of said cell membrane order.

2. A method as defined in claim 1, wherein said fluorescence spectrum includes a first spectral band and a second spectral band, computing said spectral signature including computing a first intensity of said fluorescence spectrum in said first spectral band and a second intensity of said fluorescence spectrum in said second spectral band.

3. A method as defined in claim 2, wherein computing said spectral signature includes computing a ratio between said first intensity and said second intensity.

4. A method as defined in claim 2, wherein said first spectral band is centered on a first central wavelength and said second spectral band is centered on a second central wavelength, said first central wavelength being comprised in an interval of from about 500 nm to about 600 nm and said second central wavelength being comprised in an interval of from about 650 nm to about 750 nm.

5. (canceled)

6. A method as defined in claim 2, wherein said first spectral band and said second spectral band have a first bandwidth and a second bandwidth, respectively, said first bandwidth and said second bandwidth being of about 25 nm to about 100 nm.

7. (canceled)

8. (canceled)

9. A method as defined in claim 1, wherein said fluorescence spectrum defines five spectral bands centered respectively on a respective central wavelength of about 530 nm, about 560 nm, about 585 nm, about 615, nm and about 645 nm and having respective bandwidths of about 30 nm, about 30 nm, about 20 nm, about 30 nm and about 30 nm, said fluorescence spectrum defining also a sixth spectral band including wavelengths longer or equal than or equal to about 660 nm, said five spectral bands and said sixth spectral band defining together six spectral bands, computing said spectral signature including computing a respective intensity of said fluorescence spectrum in at least three of said six spectral bands.

10. (canceled)

11. A method as defined in claim 9, wherein computing said spectral signature includes computing a respective intensity of said fluorescence spectrum in all of said six spectral bands.

12. (canceled)

13. A method as defined in claim 11, wherein computing said spectral signature includes computing all pairwise ratios between respective intensities of said fluorescence spectrum in said six spectral bands.

14. (canceled)

15. (canceled)

16. (canceled)

17. (canceled)

18. (canceled)

19. (canceled)

20. (canceled)

21. (canceled)

22. A method as defined in claim 1, wherein irradiating said stained cell with said excitation light includes irradiating said stained cell with laser light having a wavelength between about 400 nm and about 500 nm.

23. (canceled)

24. (canceled)

25. A method as defined in claim 1, wherein said spectral signature is indicative a cholesterol content of a membrane of said cell.

26. A method as defined in claim 1, wherein said spectral signature is indicative of a lipid and protein content of a membrane of said cell.

27. A method as defined in claim 1, wherein said cell is classifiable as belonging to a specific cell category selected from a set of predetermined cell categories, said method further comprising classifying said cell as belonging to said specific cell category on a basis of said spectral signature.

28. A method as defined in claim 27, wherein said set of predetermined cell categories includes cell categories indicative of a cholesterol content in said cell.

29. A method as defined in claim 27, wherein said set of predetermined cell categories includes cell categories indicative of a lipid and protein content of a membrane of said cell.

30. A method as defined in claim 27, wherein said cell is a blood platelet, said set of predetermined cell categories including cell categories indicative of a coagulation activity of said platelets.

31. A method as defined in claim 27, wherein said set of predetermined cell categories includes cell categories indicative of an apoptosis status of said cell.

32. A method as defined in claim 27, wherein said set of predetermined cell categories includes sub-populations of cells of a predetermined type.

33. A method for assessing an effect of a treatment in a subject, said treatment influencing target cells, said method comprising:
obtaining a first sample from said subject, said first sample including said target cells;
characterizing cell membrane order in said target cells of said first sample using said method as defined in claim 1;
treating said patient with said treatment;
obtaining a second sample from said subject after said treatment, said second sample including said target cells;
characterizing cell membrane order in said target cells of said second sample using said method as defined in claim 1;
assessing said effect of said treatment by comparing said cell membrane order in said target cells of said first and second samples.

34. A method as defined in claim 33, wherein said treatment is an anticoagulant treatment and said target cells are platelets, said effect of said treatment being detectable through an increase in said cell membrane order in said platelets.

35. A method as defined in claim 33, wherein said treatment includes administering to said patient a statin or clopidogrel.

36. (canceled)

37. (canceled)

38. (canceled)

39. (canceled)

40. (canceled)

41. (canceled)

42. (canceled)
